(19) United States
(12) Patent Application Publication
(10) Pub. No.: US 2008/0173310 A1
(43) Pub. Date: Jul. 24, 2008

(54) Apparatus for securing a tracheal tube or the like to a patient

(76) Inventor: Gregory P. Marcoe, Midland, MI (US)

Correspondence Address: Timothy S. Stevens, 5108 Foxpoint Circle, Midland, MI, 48642, US

(21) Appl. No.: 11/656,213

(22) Filed: Jan. 22, 2007

(51) Int. Cl. A61M 16/04

(52) U.S. Cl. 128/207.17

(57) ABSTRACT

An elongate strip of adhesive tape having an adhesive side and a non-adhesive side; (b) a central peel-away tab on and covering the adhesive side of the central portion of the adhesive tape; and (c) a pair of peel-away tabs on and covering the adhesive side of each end portion of the adhesive tape so that when the central peel-away tab is peeled off the tape and the adhesive side of the central portion of the adhesive tape is wrapped around a tracheal tube or the like, the pair of peel-away tabs on and covering the adhesive side of each end portion of the adhesive tape can be peeled off against the cheeks of the patient so that the adhesive side of each end portion of the adhesive tape adheres to the patients cheeks thereby securing the tube to the patient. In addition, an improved kit of components for securing a tracheal tube or the like to a patient, the kit of components contained in a sealed and steralizable pouch, wherein the kit includes: (a) an elongate strip of adhesive tape having an adhesive side and a non-adhesive side; (b) a central peel-away tab on and covering the adhesive side of the central portion of the adhesive tape; and (c) a pair of peel-away tabs on and covering the adhesive side of each end portion of the adhesive tape. Additional components of the kit may include eye tapes and tape for securing nasogastric tubes or the like to the patient.

### BACKGROUND OF THE INVENTION

The instant invention relates to apparatus comprising adhesive tape for securing a tracheal tube or the like to a patient.

Various apparatus comprising adhesive tape are available for securing a tracheal tube or the like (such as an airway adjunct tube, an oropharyngeat tube, a nasogastric tube or a laryngeal tube) to a patient. For example, U.S. Pat. No. 3,927,676 discloses tape having a central non-adhesive portion extending around the back of the neck of the patient, the tape having bifurcated adhesive coated ends for wrapping around a tracheal tube and then attachment back to the tape to secure the tracheal tube to the patient. U.S. Pat. No. 5,038,778 and U.S. Pat. No. 5,306,233 disclose tape having a central non-adhesive portion extending around the back of the neck of the patient, the tape having bifurcated adhesive coated ends for wrapping around a tracheal tube and attachment to the patient to secure the tracheal tube to the patient. U.S. Pat. No. 5,546,938 discloses tape having a central non-adhesive portion extending around the back of the neck of the patient, the tape having bifurcated adhesive coated ends for wrapping around a tracheal tube and attachment to the upper lip to secure the tracheal tube to the patient. U.S. Pat. No. 5,638,814 discloses tape attached to central non-adhesive strap extending around the back of the neck of the patient, the tape having a central adhesive coated X-shaped portion for wrapping around a tracheal tube to secure the tracheal tube to the patient.

Despite the advances made in the art of apparatus and methods for securing a tracheal tube or the like to a patient, such apparatus and methods have not supplanted the use of un-sterilized rolls of adhesive tape even though the use of such tape may provide a means for cross-contamination and infection. Therefore, there remains a need for apparatus and methods for securing a tracheal tube or the like to a patient that are easily used and which reduce the possibility for cross-contamination and infection.

### SUMMARY OF THE INVENTION

The instant invention provides an apparatus and method for securing a tracheal tube or the like to a patient that is easily used, which can be packaged in a sealed and sterilized single-use package thereby reducing the possibility for cross-contamination and infection. More specifically, the instant invention in one embodiment is an apparatus for securing a tracheal tube or the like to a patient, comprising: (a) an elongate strip of adhesive tape having an adhesive side and a non-adhesive side; (b) a central peel-away tab on and covering the adhesive side of the central portion of the adhesive tape; and (c) a pair of peel-away tabs on and covering the adhesive side of each end portion of the adhesive tape.

In another embodiment, the instant invention is an improved kit of components for securing a tracheal tube or the like to a patient, the kit of components contained in a sealed and steralizable pouch, wherein the improvement comprises an improved adhesive tape, comprising: (a) an elongate strip of adhesive tape having an adhesive side and a non-adhesive side; (b) a central peel-away tab on and covering the adhesive side of the central portion of the adhesive tape; and (c) a pair of peel-away tabs on and covering the adhesive side of each end portion of the adhesive tape.

In yet another embodiment, the instant invention is an improved method for securing a tracheal tube or the like to a patient using an elongate strip of adhesive tape, comprising the steps of: (a) peeling a central peel-away tab on and covering the adhesive side of a central portion of an elongate strip of adhesive tape having an adhesive side and a non-adhesive side, the adhesive tape additionally having a pair of peel-away tabs on and covering the adhesive side of each end portion of the adhesive tape; (b) wrapping the adhesive side of the central portion of the adhesive tape around the tube; (c) peeling the pair of peel-away tabs on and covering the adhesive side of each end portion of the adhesive tape against the cheeks of the patient so that the adhesive side of each end portion of the adhesive tape adheres to the patients cheeks thereby securing the tube to the patient.

### BRIEF DESCRIPTION OF THE DRAWINGS

FIG. 1 is a side view in full of an apparatus embodiment of the instant invention showing three peel-away tabs on and covering the adhesive side of an elongate strip of adhesive tape;

FIG. 2 is a perspective view in full of the apparatus shown in FIG. 1;

FIG. 3 is a front view in full of a portion of the apparatus shown in FIG. 1 after the central peel-away tab has been removed and the central portion of the apparatus is wrapped around a tracheal tube or the like;

FIG. 4 is a front view in full of the system shown in FIG. 3 in position to pull the remaining peel-away tabs from the tape ends;

FIG. 5 is a front view in full of the system shown in FIG. 4 as the remaining peel-away tabs are pulled from the tape ends to adhere the tape ends to the patient's cheeks:

FIG. 6 is a side cross-sectional view of a kit of components for securing a tracheal tube or the like to a patient, the kit of components contained in a sealed and steralizable pouch and including the apparatus of FIG. 1;

FIG. 7 is an upper view in full of the kit of components of FIG. 6 showing additional tape apparatus useful when securing a tracheal tube or the like to a patient.

### DETAILED DESCRIPTION

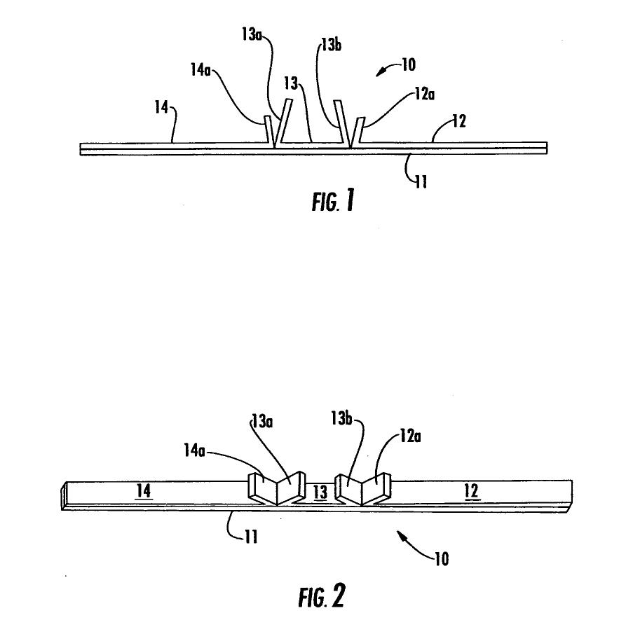

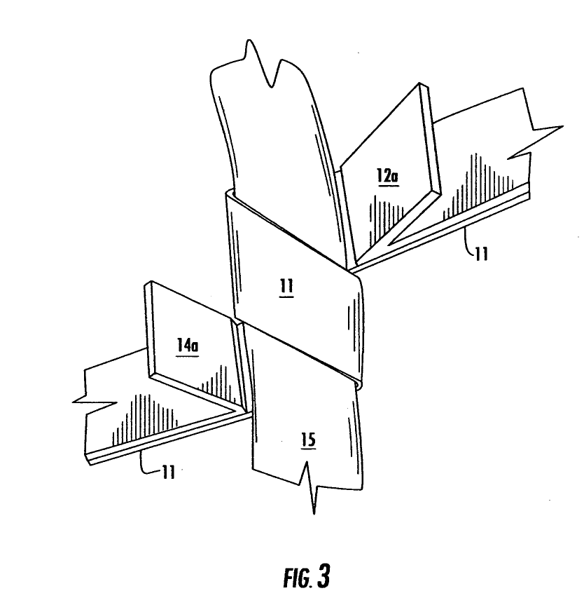

Referring now to FIG. 1, therein is shown a side view in full of an apparatus embodiment 10 of the instant invention. The apparatus 10 comprises an elongate strip of adhesive tape 11 having an adhesive side and a non-adhesive side. A central peel-away tab 13 is positioned on and covering the adhesive side of the central portion of the adhesive tape 11. The central peel-away tab 13 preferably has tab ends 13a and/or 13b so that the tab 13 can be more easily peeled off the central portion of the tape 11. A pair of peel-away tabs 12 and 14 are positioned on and covering the adhesive side of each end portion of the adhesive tape 11. The pair of peel-away tabs 12 and 14 preferably have tab ends 12a and 14a so that the tabs 12 and 14 can be more easily peeled off the end portions of the tape 11. Referring now to FIG. 2, therein is shown a perspective view of the apparatus 10 of FIG. 1.

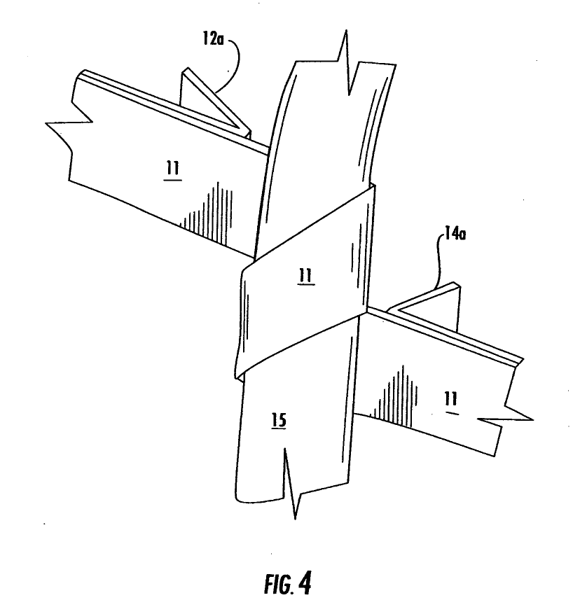

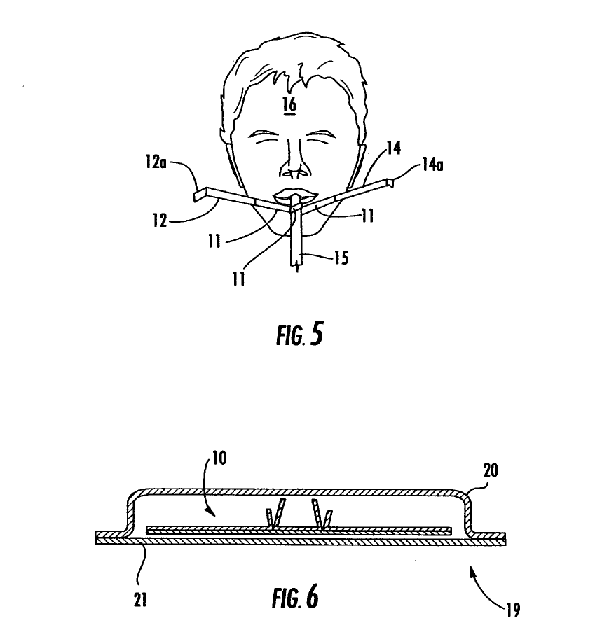

Referring now to FIG. 3, therein is shown a front view in full of a portion of the apparatus 10 shown in FIGS. 1 and 2 after the central peel-away tab has been removed and the central portion of the tape 11 is wrapped around a tracheal tube 15. Referring now to FIG. 4, therein is shown a front view in full of the system shown in FIG. 3 in position to pull the remaining peel-away tab ends 12a and 14a from the tape 11. Referring now to FIG. 5, therein is shown front view in full of the system shown in FIG. 4 as the peel-away tabs 12 and 14 are pulled from the tape 11 to adhere the ends of the tape 11 to the patient's cheeks thereby securing the tracheal tube to the patient 16.

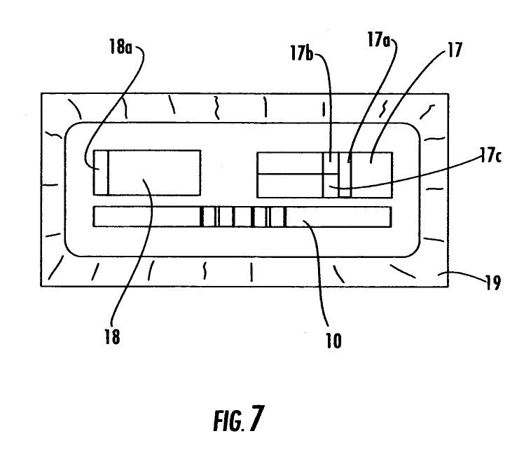

Referring now to FIG. 6, therein is shown a side cross-sectional view of a kit of components 19 for securing a tracheal tube to a patient, the kit of components contained in a conventional sealed and steralizable pouch comprised on an upper formed transparent plastic film portion 20 sealed at the edges thereof to a lower paper portion 21, the kit 19 including the apparatus 10 of FIG. 1. Referring now to FIG. 7, therein is shown an upper view in full of the kit of components 19 of FIG. 6 showing additional tape apparatus 17 and 18 useful for securing nasogastric tubes to a patient and as eye tapes. Apparatus 17 is comprised of a piece of adhesive tape having an adhesive and non-adhesive sides and a bifurcated end. Peel-away tabs 17a, 17b and 17c are positioned on and covering the adhesive side of the adhesive tape. Apparatus 18 is simply comprised of a piece of adhesive tape having adhesive and non-adhesive sides. Peel-away tab 18a is positioned on and covering the adhesive side of the adhesive tape. The kit of components 19 can be sterilized and stored for use.

The adhesive tape used in the instant invention is preferably of the type suited for attachment to the skin, which can be sterilized by conventional means (such as exposure to ethylene oxide) and which will release the peel-away tabs without undue force. Such adhesive tape is well-known in the art. The peel-away tabs are preferably made of plastic coated paper and are also well-known in the art.

### CONCLUSION

While the instant invention has been described above according to its preferred embodiments, it can be modified within the spirit and scope of this disclosure. This application is therefore intended to cover any variations, uses, or adaptations of the instant invention using the general principles disclosed herein. Further, the instant application is intended to cover such departures from the present disclosure as come within the known or customary practice in the art to which this invention pertains and which fall within the limits of the following claims.

What is claimed is:

1. Apparatus for securing a tracheal tube or the like to a patient, comprising: (a) an elongate strip of adhesive tape having an adhesive side and a non-adhesive side; (b) a central peel-away tab on and covering the adhesive side of the central portion of the adhesive tape; and (c) a pair of peel-away tabs on and covering the adhesive side of each end portion of the adhesive tape.

2. An improved kit of components for securing a tracheal tube or the like to a patient, the kit of components contained in a sealed and steralizable pouch, wherein the improvement comprises an improved adhesive tape, comprising: (a) an elongate strip of adhesive tape having an adhesive side and a non-adhesive side; (b) a central peel-away tab on and covering the adhesive side of the central portion of the adhesive tape; and (c) a pair of peel-away tabs on and covering the adhesive side of each end portion of the adhesive tape.

3. An improved method for securing a tracheal tube or the like to a patient using an elongated strip of adhesive tape, comprising the steps of: (a) peeling a central peel-away tab on and covering the adhesive side of a central portion of an elongate strip of adhesive tape having an adhesive side and a non-adhesive side, the adhesive tape additionally having a pair of peel-away tabs on and covering the adhesive side of each end portion of the adhesive tape; (b) wrapping the adhesive side of the central portion of the adhesive tape around the tube; (c) peeling the pair of peel-away tabs on and covering the adhesive side of each end portion of the adhesive tape against the cheeks of the patient so that the adhesive side of each end portion of the adhesive tape adheres to the patients cheeks thereby securing the tube to the patient.
